(19) United States
(12) Patent Application Publication
(10) Pub. No.: US 2007/0227063 A1
(43) Pub. Date: Oct. 4, 2007

(54) Process for conversion of mushroom lignocellulosic waste to useful byproducts

(75) Inventors: Bruce E. Dale, Mason, MI (US); Venkatesh Balan, East Lansing, MI (US); Shishir P.S. Chundawat, East Lansing, MI (US)

Correspondence Address: Ian C. McLeod;IAN C. MCLEOD, P.C., 2190 Commons Parkway, Okemos, MI, 48864, US

(73) Assignee: Board of Trustees of Michigan State University, East Lansing, MI (US)

(21) Appl. No.: 11/729,632

(22) Filed: Mar. 29, 2007

Related U.S. Application Data
(60) Provisional application No. 60/787,595, filed on Mar. 30, 2006.

(51) Int. Cl. A01G 1/04; C12P 7/06

(52) U.S. Cl. 47/1.1; 435/161

(57) ABSTRACT

A process for the conversion of monocot lignocellulosic grass waste from mushroom growth into byproducts is described. In particular, the present invention releases glucans from the waste which can be easily hydrolyzed, after a less severe thermochemical process (i.e. AFEX), and into sugars for producing ethanol or other by-products by fermentation.

### CROSS-REFERENCE TO RELATED APPLICATIONS

This application claims benefit to U.S. Provisional Application Ser. No. 60/787,595, filed Mar. 30, 2006, which is incorporated herein by reference in its entirety.

### STATEMENT REGARDING GOVERNMENT RIGHTS

Not Applicable.

### BACKGROUND OF THE INVENTION

(1) Field of the Invention

The present invention relates to a process for the conversion of mushroom waste grasses into free sugars in high yield. In particular, the present invention relates to a process wherein the waste grass is rice straw used for the growth of mushrooms such as Pleurotus ostreatus.

(2) Description of the Related Art

There is a growing need to find replacements for petroleum, a depleting non-renewable resource, as the primary feedstock for the chemicals and fuels industry. Ethanol has many desirable features as a petroleum substitute and could help make a smoother transition from a petroleum-based to a bio-based chemical industry. Ethanol is produced in large quantities from natural resources like corn grain and sugarcane juice. However, there is need to find an inexpensive and widely available lignocellulosic source of biomass (i.e. corn stover, rice straw, wheat straw etc.) to avoid feedstock conflict with the prevalent food industry. Lignocellulosic compositional recalcitrance is one of the primary impediments in the successful implementation of an ethanol based biorefinery. Pretreatment of biomass to reduce this intrinsic recalcitrance becomes critical to help improve bioconversion.

Rice straw is an important lignocellulosic biomass with nearly 900 million dry tons produced annually worldwide. Rice straw therefore has great potential for making renewable fuels. However, rice straw is a more recalcitrant lignocellulosic material that requires some form of severe thermochemical pretreatment to avail high hydrolysis and fermentation yields (Gollapalli et al., 2002). For example, about two thirds of ammonia fiber expansion (AFEX) pretreated rice straw glucan was hydrolyzed to glucose, while we see almost 95% glucan conversion for AFEX treated corn stover under similar hydrolysis conditions.

Fungal growth on lignocellulosics has been known for several centuries for producing edible mushroom (Israilides et. al, 2003). Currently, there are over 3 million metric tones of edible mushrooms produced in 2002 (with over 200 species); in the world using a wide variety of biomass (Poppe 2000, USDA report). The world market for the mushroom industry in 2005 was valued at over $45 billion (Shu-Ting Chang, 2006). Rice straw is one of the biomass which is extensively used for growing Oyster mushrooms, next to composted wood chips (Zang et. al., 2002, Obodai et. al., 2003). A large amount of Mushroom Spent Straw (MSS) is currently generated during the process and are used for various purposes like burning (Williams, et. al., 2001), land fill, compost (Singh et. al., 2002), animal feed (Karunanandaa et. al., 1995, Sanchez et. al., 2002) and for making bio-plastics (Houghton et. al., 2004).

Biological pretreatment of lignocellulosics is an area that has been looked at very closely in recent years due to several advantages that it offers to improve the quality of feedstock (Martinez et. al., 2005) for downstream enzymatic hydrolysis and fermentation. Some of the most important advantages of biological pretreatment being the lower energy requirements for the process, higher yields and no inhibitors produced to cellulosic hydrolysis and fermentation (Keller et al., 2003). The most promising microbes for biological pretreatment are white-rot fungi which can enhance enzymatic hydrolysis of biomass (Taniguchi et. al., 2005; Cohen et. al., 2002). Further, they found improvements in nutritive value of biomass and are used as animal feeds (Karunanandaa et. al., 1995).

Many of the present-day biomass pretreatments (ethanol organosolv pretreatment, dilute acid pretreatment, AFEX, lime pretreatment, ammonia recycle percolation) fractionate the various biomass components (lignin, hemicellulose & cellulose) into separate process streams (Mosier et. al., 2005). The removal of lignin and/or hemicellulose can substantially reduce the recalcitrance of biomass to enzymatic hydrolysis (Wyman et al., 2005). However, the thermochemical pretreatment fractionation method is rather energy intensive and generates waste streams making it a significant bottleneck to an economical bioconversion process. Ammonia fiber explosion (AFEX) pretreatment (Dale, 1986) is a novel alkaline pretreatment process that effectuates a physico-chemical alteration in the lignocellulosic ultra and macro structure. Studies have shown that the AFEX pretreatment helps to increase enzymatic digestibility several folds over the untreated lignocellulosic (Teymouri et. al., 2005). AFEX pretreatment results in the decrystallization of cellulose (Gollapalli et al., 2002), partial depolymerization of hemicellulose, deacetylation of acetyl groups (O'Connor, 1972), cleavage of lignin carbohydrate complex (LCC) linkages, lignin C—O—C bond cleavage, increase in accessible surface area due to structural disruption (Turner et al., 1990) and increased wettability of the treated biomass (Ferrer et. al., 1999). The AFEX process demonstrates attractive economics compared to several leading pretreatment technologies based on a recent economic model (Eggeman et al., 2005) for bio-ethanol from corn stover.

AFEX pretreatment has proved to be quite ineffective for hardwood, softwood and more recalcitrant lignocellulosics like bagasse and rice straw. One possible reason for this is the tougher lignin seal provided in these harder lignocellulosics that prevent effective diffusion and reaction of ammonia into the ultra structure. In order to make AFEX more effective for such highly recalcitrant species one could carry out some sort of structural modification of the biomass to aid the subsequent ammonia treatment process.

Biological pretreatment of lignocellulosics is an area that has been looked at very closely in recent years due to several advantages that it offers (Keller et al., 2003). Some of the most important advantages of biological pretreatment being the lower energy requirements for the process, higher yields and no inhibitors produced to cellulosic hydrolysis and fermentation. The most promising microbes for biological pretreatment are white-rot fungi that enhance enzymatic hydrolysis of biomass and improve its nutritive value (Cohen et. al., 2002).

### OBJECTS

It is an object of the present invention to produce an economical biorefinery process coupled with the biological pretreatment of lignocellulosic grasses to make them more amenable to AFEX treatment. Further, it is an object of the present invention to provide a process which is economical and provides value addition for by-products of the existing mushroom industry.

These and other objects will become increasingly apparent by reference to the following description and the drawings.

### SUMMARY OF THE INVENTION

The present invention relates to a process for conversion of monocot lignocellulosic grasses used in the growth of mushrooms into byproducts which comprises:

(a) growing and harvesting edible mushrooms in a grass so that lignins are degraded, wherein a liquid and a solid as byproducts are produced from the growth of the mushrooms;

(b) separating the liquid which is water soluble from the solids, wherein the liquid comprises enzymes and degraded lignin produced during the growth of the mushrooms on the grass;

(c) treating the solid separated from the liquid with an AFEX process using pressurized hot liquid ammonia which is rapidly depressurized to release the ammonia treated solid as the byproduct;

(d) optionally treating the ammonia treated solid as the byproduct with the glucans from step (c) with a cellulase and β-glucosidase enzyme mixture to produce sugars; and

(e) optionally fermenting the sugars to step (d) to produce ethanol and other chemicals.

Preferably, wherein the mushroom is Pleurotus ostreatus. Most preferably, wherein the grass is rice straw. Further, wherein the mushrooms are grown to a first harvest of the edible mushrooms in step (a). Still further, wherein liquid is removed by filtration in step (b). Further, wherein the ammonia in step (c) is at a pressure of about 100 to 450 psi and a temperature of about 30 to 180° C. Still further, wherein in step (b), the enzymes are isolated from the liquid. Furthermore, wherein in step (b), the enzyme liquid is isolated by membrane filtration. Still further, wherein in step (c), the released ammonia is recycled. Further, wherein the solids of step (a) are washed with water in step (b) for separating the liquid from the solid. Still further, wherein step (d) is performed. Finally, wherein steps (d) and (e) are performed.

The substance and advantages of the present invention will become increasingly apparent by reference to the following drawings and the description.

### BRIEF DESCRIPTION OF THE DRAWINGS

FIGS. 1A and 1B are graphs showing harvest period and the substrate composition on different days during mushroom growth. Here, FIG. 1A shows how mushroom (dwb-dry weight basis) are harvested at three stages at different intervals in a commercial mushroom farm by maintaining sterile rice straw (1 kg on dwb) with mushroom spawns in a polythene bag at 70° F. and 85% moisture conditions. FIG. 1B shows changes in percentage composition of components of rice straw after pretreatment with Pleurotus ostreatus at different days. Components: HL, hemicellulose; C, cellulose; ASL, acid soluble lignin; KL, Klason lignin; O, others (mainly ash); WL, weight loss after pretreatment. FIG. 1B reproduced with permission from Taiguchi et al., J. Biosci. Bioeng. 100, 637-643 (2005).

FIG. 2 is a chart showing overall mass balance during the washing step for rice straw (RS) and Mushroom Spent Straw (MSS). Liquid stream was further separated into two streams namely retentate and filtrate using membrane filtration (MF) with >10 kDa.

FIGS. 3A and 3B are enzymatic hydrolysis of Rice Straw (RS) and Mushroom Spent Straw (MSS). FIG. 3A shows glucan conversion and FIG. 3B shows xylan conversion varying AFEX conditions.

FIGS. 4A, 4B, 4C and 4D are photographs showing untreated and AFEX treated Rice Straw (RS) and Mushroom Spent Straw (MSS). FIGS. 4A and 4B show untreated Rice Straw (RS) and AFEX treated RS (AFEX-RS), respectively. FIGS. 4C and 4D show untreated MSS and AFEX treated MSS (AFEX-MSS), respectively. The MSS and AFEX-MSS are darker, softer and more fragile compared to the untreated RS and AFEX-RS, respectively.

### DESCRIPTION OF THE PREFERRED EMBODIMENTS

Rice straw (RS) is an important lignocellulosic biomass with nearly 900 million dry tons produced annually worldwide. Rice straw has great potential as lignocellulosic feedstock for making renewable fuels and chemicals in a biorefinery. However, due to its natural recalcitrance, rice straw needs thermochemical treatment prior to further biological processing. Ammonia Fiber Expansion (AFEX) is a leading biomass pretreatment process utilizing concentrated/liquefied ammonia to pretreat lignocellulosic biomass at moderate temperatures (70-120° C.). Previous research has shown improved cellulose and hemicellulose conversions upon AFEX treatment of rice straw at 2:1 ammonia to biomass (w/w) loading, 40% moisture (dwb) and 90° C. However, there is still room for much improvement.

Fungal pretreatment of lignocellulosics is an important biological pretreatment method that has not received much attention in the past. This is probably due to the long residence time of this process and also reduces overall yield. However, the overall sugar loss may be minimized through use of white-rot fungi (i.e. Pleutorus ostreatus) over a much shorter incubation time, if combined with a subsequent low severity thermochemical pretreatment step. It was found that Mushroom Spent Straw (MSS) with AFEX allowed reduction in pretreatment severity, while giving higher glucan conversions (up to 25%) than before. In this invention, the effect of fungal conditioning of rice straw on AFEX pretreatment and enzymatic hydrolysis is described. The recovery of other byproducts from the fungal pretreatment process such as fungal enzymes, mushrooms, oligosaccharides, organic acids, and the like are also disclosed.

Materials and Methods

Water Washing of Untreated/AFEX Treated Biomass

Untreated and AFEX treated biomass were presoaked and washed in distilled (de-ionized) water with a substrate to water loading of 1:10 (w/w). The slurry was mixed for 15 minutes. The wash liquid was removed from the substrate by squeezing the slurry through a filtration cloth (Calbiochem, CA) and stored in the refrigerator for further analysis. The washed substrates were enzymatically hydrolyzed immediately to prevent microbial growth. The moisture content of the washed substrate was determined using a moisture analyzer (Model MF-50, A&D).

Mass Balance for Water Washing

A mass balance for determining the solids and soluble content of the wash liquid stream was carried out according to the procedure outlined before. Untreated Rice Straw (RS) and Mushroom Spent Straw (MSS) (200 gms dry BM each) were soaked and stirred in distilled (de-ionized) water with a substrate to water loading of 1:10 for 15 minutes. The wash liquid was removed from the substrate by filtering the slurry through a filtration cloth (Calbiochem, CA) and centrifuged at 9000 rpm to remove fine solid particles from the wash stream, then concentrated using membrane filtration (10 kDa). The supernatant was lyophilized to recover the washed soluble from the biomass for further characterization. The moisture content of the sample was determined after keeping the sample for 12 h in a lab oven at 105° C.

Composition Analysis:

Lignin, sugar and protein content in the biomass were measured by dairy one (New york, Ithaca) using standard analytical protocols. The composition analysis were done for Rice Straw (RS) and Mushroom Spent Straw (MSS) using NREL protocol (LAP001, 002 and 012) using dilute acid followed by HPLC analysis as given below.

AFEX Pretreatment

Mushroom Spent Straw (MSS) and Rice Straw (RS) were pretreated by the AFEX pretreatment process. The biomass with varying moisture (10-60%) (kg water/kg dry biomass) was transferred to a high-pressure Parr reactor and liquid ammonia (1 kg of ammonia/kg of dry biomass) was slowly charged to the vessel. The temperature was raised and maintained at 90° C. for five minutes residence time at that temperature before explosively relieving the pressure. Overall, it took approximately 30-40 minutes to complete one cycle of pretreatment process. The instantaneous drop of pressure in the vessel caused the ammonia to vaporize, causing an immediately decompression of the biomass and considerable fiber disruption. The pretreated material was allowed to stand under a hood overnight to remove the residual ammonia and stored in a freezer until further use.

Enzymatic Hydrolysis

The NREL standard protocol (LAP-009) was followed for enzymatic hydrolysis of the biomass. Cellulase (Spezyme CP) enzyme was a generous gift from Genencor International (Rochester, N.Y.). The substrate was hydrolyzed at a glucan loading of 1% (w:v) in a 0.05 molar citrate buffer solution (pH 4.8) at the desired cellulase enzyme loading (protein concentration 123 mg/ml) of 15 FPU/gm glucan and β-glucosidase (Sigma, St. Louis, Mo.) loading of 64 pNPGU/gm glucan. Xylanase (protein concentration 42 mg/ml) supplementation was carried out at 10% of the total milligrams of cellulase protein loaded. The protein concentration of the enzymes was determined by the BCA protein assay (Pierce, Rockford, Ill.). Samples were hydrolyzed at 50° C. with gentle agitation (90 rpm) for a period of 168 hours. The hydrolyzed samples were boiled to denature the enzymes and filtered through a 0.2 micron nylon membrane filter at predetermined time periods (72 and 168 hours). The samples were frozen for subsequent HPLC sugar analysis.

HPLC Sugar Analysis

A high performance liquid chromatography (HPLC) system was used for sugar analysis. The HPLC system consisted of Waters (Milford, Mass.) Pump and Waters 410 refractive index detector, an Aminex HPX-87P carbohydrate analysis column (BioRad, Hercules, CA) equipped with a de-ashing guard cartridge (BioRad). Degassed HPLC grade water was used as the mobile phase at 0.6 ml/min at a column temperature of 85° C. The injection volume was 20 μl with a run time of 20 min. Mixed sugar standards were used for quantification of cellobiose and other monosaccharides (glucose, xylose, galactose, arabinose and mannose) in the samples.

Results and Discussions

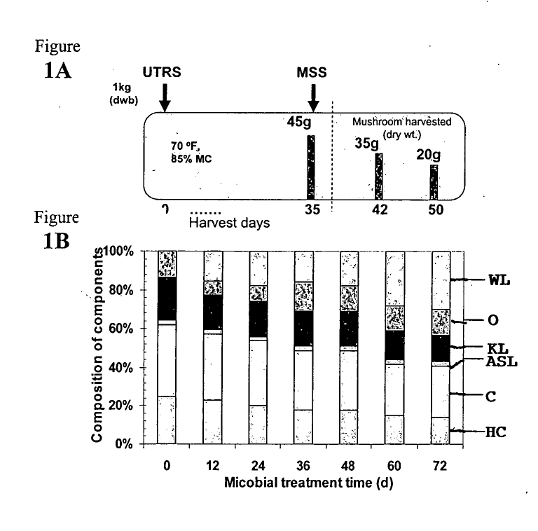

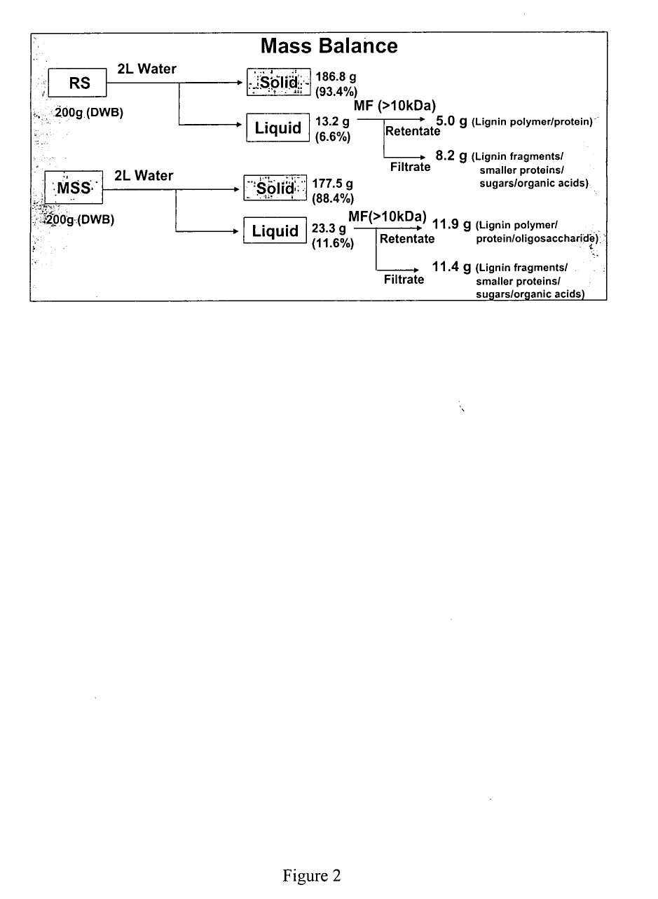

With the growing demand for ethanol, biorefinery using lignocellulosic feed stock can be successfully implemented depending on its large scale availability of biomass on a regular basis. Since corn starch and cane sugar ethanol plants are well established, lignocellulosic waste generated from these plants can as well be transported and will be used for making ethanol in the near future. Logistic study of procuring, transporting and storing these materials are underway. A constant source of lignocellulosic feed stock is Mushroom Spent Straw (MSS) from a mushroom plant (Obodi et. al, 2003). In 2005, the world market for mushroom is $41 billion (Shu-Ting Chang, 2006). Over 4-5 million tons of edible mushrooms are produced worldwide using different substrates. Harvest period and substrate compositions on different days during mushroom growth are given in FIGS. 1A and 1B. Traditionally, oyster mushrooms are cultivated using sterile rice straw packed in a polythene bag with spawns distributed at regular intervals followed by storing the bag in humid environment for a period of 50 days. About 100 g mushroom (dwb) can be harvested at three intervals starting from 1 kg rice straw (dwb). In order to conserve both lignin and hemicellulose for getting maximum sugars, microbial conditioning was stopped after 35 days. Further, Mushroom Spent Straw (MSS) was extracted with water to remove enzymes/proteins, organic acids, soluble lignin and oligosaccharides. The higher molecular weight components (>10 kDa) namely enzymes, soluble lignin polymer and oligosaccharides were removed using membrane filtration as retentate. While the low molecular weight components like lignin degradation products, organic acids, lower molecular weight enzymes/proteins were obtained as filtrate. The detailed mass balance for the above washing protocol is given in FIG. 2.

There are several advantages in using the Mushroom Spent Straw (MSS) because it is available in plenty in pre-processed form. In other words, the microbes degrade the lignin using various combinations of enzymes leaving behind the hemicellulose and cellulose which could be easily accessed during enzymatic hydrolysis. Potential for reducing severity of thermo chemical biomass pretreatment was demonstrated for microbial pretreated biomass (Keller et. al., 2003). A substantial amount of composition change (FIGS. 1A and 1B) was noticed during the process (Taniguchi et. al., 2005). A combination of key enzymes like, lacases, manganese peroxidase, lignin peroxidase and phenol oxidase were secreted by white rot fungus (Table 1) and synergistically degrade lignin (Martinez et. al., 2005). Since lignin is the most recalcitrant molecule during the enzymatic hydrolysis, removing them prior to hydrolysis would be expected to provide higher sugar yields. As expected, when Mushroom Spent Straw (MSS) was hydrolyzed, we could obtain a sugar conversion of up to 40% using 15 FPU of commercial cellulase enzymes, compared to just 20% for untreated rice straw. Hence, a second pretreatment is required for getting even higher sugar yields.

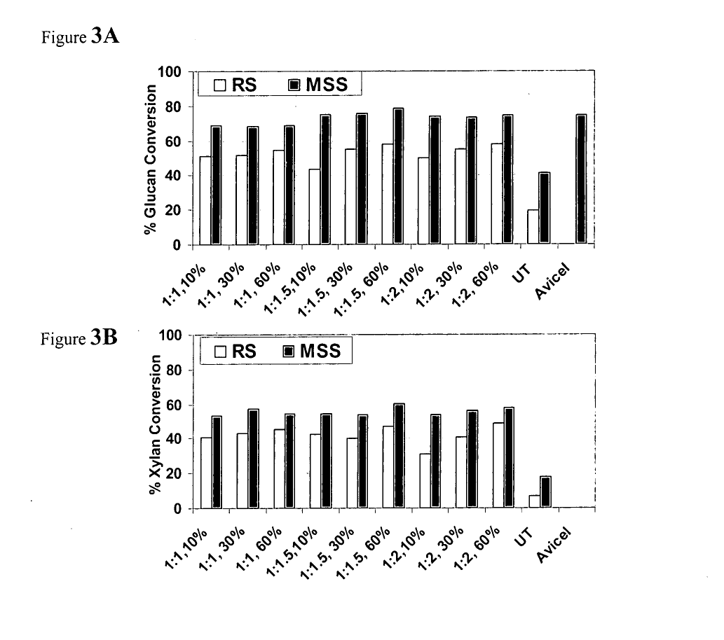

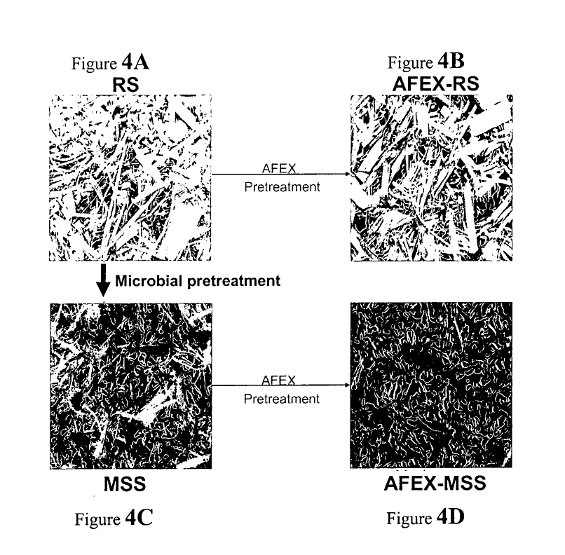

In one of our previous studies, pretreatment of rice straw using AFEX (2:1 ammonia to biomass, 40% moisture, 90° C.) could achieve maximum 70-80% glucan conversion (Gollapali et al., 2002) using a much higher cellulase loading of 75 FPU/gm glucan. In the new approach, we used Mushroom Spent Straw (MSS) using Pleurotus ostreatus strain after washing in water, followed by AFEX pretreatment (at fixed temperature of 90° C., varying biomass to ammonia ratios from 1:1 to 1:2 and varying moistures) to achieve close to 80% glucan conversion using just 15 FPU/g glucan loading of cellulase enzyme in under 72 hours (FIGS. 3A and 3B). Picture showing both Rice Straw (RS) and Mushroom Spent Straw (MSS), before and after AFEX treatment is given in FIGS. 4A to 4D. Less severe AFEX conditions are required to achieve the same conversions for Mushroom Spent Straw (MSS). Increasing enzyme loading or increasing the reaction time, further improves the glucan/xylan conversions (results not shown). By the present invention, the waste material could be completely hydrolyzed by pretreatment using AFEX and further can be fermented to ethanol. A preliminary analysis on various metabolites generated during the solid state fermentation process was performed by analyzing the wash stream using HPLC. Compared to untreated rice straw, MSS treated rice straw showed around 10 different organic acids (results not shown). In addition, we can extract some enzymes as reported before (Baldrian and Gabriel, 2002; Christian et. al., 2005) which have a wide range of applications in degrading xenobiotic compounds and aromatic compounds in dye industry. At the end of the process, about 10-14% of silica was left behind which could be a valuable inorganic resource for some specific applications. Soluble lignin which was extracted along with protein and polypeptides are also considered as one of the important byproducts in the whole process. The enzymes and organic acids removable from the process are shown in Table 1.

TABLE 1

| ENZYMES | ORGANIC ACIDS |
|---|---|
| Laccase (LaC) | Acetate |
| Lignin Peroxidase (LiP) | Propionate |
| Phenol oxidizing enzymes | Formate |
| Maganese Peroxidase (MgP) | Pyruvate |
| Versatile Peroxidase (VsP) | Sorbate |
| Protease | Glutamate |
| Cellulase | Carbonate |
| Xylanase | Succinate |
| | Tartrate |
| | Phthalate |

In addition, enzyme combination studies can be used in order to improve the xylan conversion. Higher solid loading during enzymatic hydrolysis can be used followed by fermenting the sugars to ethanol using yeast strain.

### REFERENCES

1. Baldrian, P, Gabriel, J. 2002. Variability of Laccase activity in the white-rot Basidomycete Pleurotus ostreatus. Folia Microbiol. 47:385-390.
2. Cohen R, Persky, L, Hadar, Y. 2002. Biotechnological applications and potential of wood-degrading mushrooms of the genus Pleurotus. Appl. Microbiol. Biotechnol. 58:582-594.
3. Chahal, D S. 1984. Biotechnol. Bioeng. Symp., pp. 425-433.
4. Christian V, Shrivastava R, Shukla D, Modi H A and Vyas B R M. 2005. Degradation of xenobiotic compounds by lignin-degrading white-rot fungi: Enzymology and mechanism involved. Ind. J. of Exp. Biol. 43, 301-312.
5. Dale B E. 1986. Method for increasing the reactivity and digestibility of cellulose with ammonia. U.S. Pat. No. 4,600,590
6. Eggeman T, Elander R T. 2005. Process and economic analysis of pretreatment technologies. Bioresour Technol 96:2019-2025.
7. Gollapalli L E, Dale B E, Rivers D M. 2002. Predicting digestibility of ammonia fiber explosion (AFEX) treated rice straw. Appl Biochem Biotech 98-100:23-35.
8. Houghton T P, Thompson D N, Hess J R, Lacey J A, Wolcott M P, Schirp A, Englund K, Dostal D and Loge F. 2004. Fungal upgrading of wheat straw for straw-thermoplastic production. Appl. Biochem. Biotechnol. 113-116: 71-93.
9. Israilides C and Philippoussis. 2003. A Bio-technologies of recycling agro-industrial wastes for the production of commercially important fungal polysaccharides and mushrooms. Biotechnol. Genet Eng. Rev. 20:247-259.
10. Keller F A, Hamilton, Je, Nguyen Q A. 2003. Microbial pretreatment of biomass:potential for reducing severity of thermochemical biomass pretreatment. Appl. Biochem. Biotechnol. 105-108:27-41.
11. Karunanandaa K, Varga G A, Akin D E, Rigsby L L and Royse D J. 1995. Botanical fractions of rice straw colonized by white-rot fungi:changes in chemical composition and structure. Animal Feed Science Technol. 55:179-199.
12. Martinez A T, Speranza M, Ruiz-Duenas F J, Ferreira P, Camarero S, Guillen G, Martinez M J, Gutierrez A and Del-Rio J C. 2005. Biodegradation of lignocellulosic: microbial, chemical and enzymatic aspects of the fungal attack of lignin. Int. Microbiol. 8:195-204.
13. Mosier N, Wyman C, Dale B, Elander R, Lee Y Y, Holtzapple, M, Ladisch M. 2005. Features of promising technologies for pretreatment of lignocellulosic biomass. Bioresource Technol. 96:673-686.
14. Obodai M, Cleland-Okine, J, Vowotor, K A. 2003. Comparative study on the growth and yield of pleurotus ostreatus mushroom on different lignocellulosic by-products. J. Ind. Microbiol. Biotechnol. 30: 146-149.
15. O'Connor J J. 1972. Tappi 55:353.
16. Poppe J. 2000. Use of agricultural waste materials in the cultivation of mushrooms. Science and Cultivation of edible fungi, Van Griensven (ed) Balkema, Rotterdam, pp. 3-23.
17. Sarikaya A and Ladisch M R. 1999. Solid-state fermentation of lignocellulosic plant residues from brassica napus by Pleurotus ostreatus. Appl. Biochem. Biotechnol. 82:1-15.
18. Sanchez A, Ysunza F, Beltran-Garcia M J, Esqueda M. 2002. Biodegradation of viticulture wastes by Pleurotus: a source of microbial and human food and its potential use in animal feeding. J Agric Food Chem. 50:2537-2342.
19. Singh A, Sharma S. 2002. Composting of a crop residue through treatment with microorganisms and subsequent vermi-composting. Bioresour. Technol. 85:107-111.
20. Ferrer A, Byers F M, Sulbaran de Ferrer B, Dale BE and Ricke S C. 1999. Increasing nutrient availability of feather meal for ruminants and non-ruminants using ammonia pressurization/depressurization process. J. Sci. Food Agric. 79:828-832.
21. Shu-Ting Chang. 2006. The World Mushroom Industry: Trends and Technological Development. Int. J. of Med. Mushrooms. 4:108.
22. Taniguchi M, Suzuki H, Watanabe D, Sakai K, Hoshino K and Tanaka T. 2005. Evaluation of Pretreatment with Pleurotus ostreatus for Enzymatic Hydrolysis of rice straw. J. Biosci. and Bioeng. 100: 637-643.
23. Teymouri F, Laureano-Perez L, Alizadeh H, Dale B E. 2005. Optimization of the ammonia fiber explosion (AFEX) treatment parameters for enzymatic hydrolysis of corn stover. Bioresour Technol 96:2014-2018.
24. Turner, N D, Byers, F M, McDonough, C M, Holtzapple, M T, Dale, B E, Jun, J H and Greene, L W, 1990, Disruption of Forage structure with an ammonia fiber explosion process, J. Animal Sci. 68:194
25. Wyman C E, Dale B E, Elander R T, Holtzapple M, Ladisch M R, Lee Y Y. 2005. Coordinated development of leading biomass pretreatment technologies. Bioresour Technol 96:1959-1966.
26. Williams B C, McMullan J T, McCahey S. 2001. An initial assessment of spent mushroom compost as a potential energy feedstock. Bioresour. Technol. 79:227-30.
27. World mushroom industry production report prepared by USDA Economics and Statistics System <www.:http://usda.mannlib.cornell.edu/data-sets/specialty/94003/>.
28. Zhang R, Li X, Fadel J G. 2002. Oyster mushroom cultivation with rice and wheat straw. Bioresour Technol. 82:277-84.
   Related Patents:
U.S. Pat. No. 5,047,332 to Chahal
U.S. Pat. No. 4,848,026 to Dunn-Coleman et al
U.S. Pat. No. 4,526,791 to Young
U.S. Pat. No. 4,370,351 to Harper
U.S. Pat. No. 4,263,744 to Stoller
U.S. Pat. No. 4,600,590 to Dale

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

We claim:

1. The present invention relates to a process for conversion of monocot lignocellulosic grasses used in the growth of mushrooms into byproducts which comprises:
(a) growing and harvesting edible mushrooms in a grass so that lignins are degraded, wherein a liquid and a solid as byproducts are produced from the growth of the mushrooms;
(b) separating the liquid which is water soluble from the solids, wherein the liquid comprises enzymes and degraded lignin produced during the growth of the mushrooms on the grass;
(c) treating the solid separated from the liquid with an AFEX process using pressurized hot liquid ammonia which is rapidly depressurized to release the ammonia treated solid as the byproduct;
(d) optionally treating the ammonia treated solid as the byproduct with the glucans from step (c) with a cellulase and β-glucosidase enzyme mixture to produce sugars; and
(e) optionally fermenting the sugars to step (d) to produce ethanol and other chemicals.

2. The process of claim 1 wherein the mushroom is Pleurotus ostreatus.

3. The process of claims 1 or 2 wherein the grass is rice straw.

4. The process of claims 1 or 2 wherein the mushrooms are grown to a first harvest of the edible mushrooms in step (a).

5. The process of claims 1 or 2 wherein liquid is removed by filtration in step (b).

6. The process of claims 1 or 2 wherein the ammonia in step (c) is at a pressure of about 450 to 100 (psi) and a temperature of about 30 to 180° C.

7. The process of claims 1 or 2 wherein in step (b), the enzymes are isolated from the liquid.

8. The process of claims 1 or 2 wherein in step (b), the enzyme liquid is isolated by membrane filtration.

9. The process of claim 1 wherein in step (c), the released ammonia is recycled.

10. The process of claims 1 or 2 wherein the solids of step (a) are washed with water in step (b) for separating the liquid from the solid.

11. The process of claims 1 or 2 wherein step (d) is performed.

12. The process of claims 1 or 2 wherein steps (d) and (e) are performed.
